(19) United States
(12) Patent Application Publication
(10) Pub. No.: US 2025/0154495 A1
(43) Pub. Date: May 15, 2025

(54) Construction Method and Sequencing Method for Single-Cell Transcriptome Sequencing Library and Kit for Preparing Single-Cell Transcriptome Library

(71) Applicant: BGI SHENZHEN, Shenzhen, Guangdong (CN)

(72) Inventors: Mingyue WANG, Shenzhen, Guangdong (CN); Longqi LIU, Shenzhen, Guangdong (CN); Chuanyu LIU, Shenzhen, Guangdong (CN); Cong YU, Shenzhen, Guangdong (CN); Yaling HUANG, Shenzhen, Guangdong (CN); Xiumei LIN, Shenzhen, Guangdong (CN); Yue YUAN, Shenzhen, Guangdong (CN); Zhifeng WANG, Shenzhen, Guangdong (CN); Xiaoyu WEI, Shenzhen, Guangdong (CN); Xiaoshan WANG, Shenzhen, Guangdong (CN); Rong XIANG, Shenzhen, Guangdong (CN)

(21) Appl. No.: 18/839,436

(22) PCT Filed: Feb. 18, 2022
(86) PCT No.: PCT/CN2022/076852

§ 371 Date: Aug. 18, 2024

(51) Int. Cl. C12N 15/10

(52) U.S. Cl. CPC C12N 15/1065

(57) ABSTRACT

Provided are a construction method and a sequencing method for a single-cell transcriptome sequencing library and a test kit for preparing a single-cell transcriptome library. In the method, a single-cell suspension is subjected to in-situ reverse transcription in a cell or a cell nucleus, the cell or the cell nucleus, together with a magnetic bead, is overloaded into a droplet for a droplet PCR reaction, and then the magnetic bead is recovered and subjected to secondary amplification, fragmentation and library construction, such that the single-cell transcriptome sequencing library is obtained. According to the method, overloading of the cell or cell nucleus subjected to the in-situ reverse transcription together with the pretreated magnetic bead is achieved, an ultra-high throughput of 100,000 cells in a single experiment is obtained, and quite high accuracy is exhibited.

### TECHNICAL FIELD

The present invention relates to the technical field of molecular biology, in particular to a construction method and a sequencing method for a single-cell transcriptome sequencing library, and a test kit for preparing the single-cell transcriptome library.

### BACKGROUND

A single cell is a fundamental unit of structures and functions in biological systems. However, conventional methods based on large number of cells for analyzing contents of molecules in biological samples mask the heterogeneity of the cells in the biological systems. The first single-cell sequencing technology emerged in 2009, and since then, more and more research has been performed at the single-cell level, and analyzing the transcriptome of a single cell has already become an effective strategy to solve the biological heterogeneity.

Since the expression levels of mRNAs in a cell are related to the functions of said cell, single-cell transcriptome sequencing can be used to classify cell types and detect cell states. Although single-cell RNA sequencing (sc-RNA-seq) methods are rapidly increasing, a traditional droplet microfluidic method for acquiring single-cell RNA sequencing data is very expensive, and its cost is scaling linearly with the number of processed cells. In addition, due to the need of avoiding cell doublets in droplets, it is necessary to adjust the cell density to a very low extent for loading the droplets. This leads to the extremely low efficiency of capturing cells in a single experiment, and the cost of the single-cell RNA sequencing also increases invisibly.

Indexing the cells in advance is a very good strategy that can be used to distinguish the cells in the same droplet. In addition, there are some methods currently that can achieve pre-indexing of RNA within cells. The most commonly used method is to reversely transcribe RNA within cell nuclei, an indexed oligothymidine (oligo-dT) primer is used to hybridize with the polyadenylate (poly A) tail of an mRNA, and then reversely transcribed into a cDNA product by using the RNA as a template under the action of a reverse transcriptase. Thereby the obtained cDNA product carries the barcode on the oligo-dT primer, and the pre-indexing of intracellular transcripts is achieved with this method.

The method of pre-indexing requires reverse transcription at the cellular or nuclear level. At present, this method is already used in some technologies. Cao et al. (Comprehensive single-cell transcriptional profiling of a multicellular organism; Science; 18 Aug. 2017: Vol. 357, Issue 6352, pp. 661-667) discloses an sci-RNA-seq technology and an sci-RNA-seq3 technology. In the two technologies, the cells are first evenly distributed into a 96- or 384-well plate, and a type of indexed oligo-dT primer is added to each well. In other words, there are a total of 96 or 384 types of barcodes in the first round of indexing. Then the same reverse transcription reagent is added to each well for a reverse transcription reaction. After the first round of indexing, all cells or cell nuclei inside the well plate are recovered. Recovered products are evenly distributed into a second 96- or 384-well plate. Similarly, a different type of indexed primer is added to each well, namely there are a total of 96 or 384 types of barcodes in the second round of indexing. In the second round, indexing is performed by PCR (sci-RNA-seq) or a ligation reaction (sci-RNA-seq3). This method of multi-round cell indexing can acquire tens of thousands of cells in a single experiment, and thus the throughput of the single experiment is greatly improved, thereby saving the cost. However, the disadvantage of this experimental method is that it is a manual operation based on the well plate, which cannot achieve automated cell capture and greatly increases the workload; in addition, its experimental steps are very cumbersome; and each round of cell recovery will cause a certain amount of loss, resulting in a relatively low recovery rate of the cells.

Another method for pre-indexing is a single-cell sequencing technology based on droplet microfluidics (drop-seq). Macosko et al. (High Parallel Genome-wide Expression Profiling of Individual Cells Using Nanoliter Droplets; Cell; May 21, 2015, Vol. 161, Issue 5, pp. 1202-1214) discloses a single-cell sequencing method using a microliter-droplet. In this method, based on a droplet microfluidics technology, a water-in-oil droplet is used to encapsulate a cell and a micro-particle, so that a droplet simultaneously containing one cell and one micro-particle is obtained. Other components in the droplet also include a lysate, and the micro-particle comprises a large number of DNA sequences with unique molecular identifiers, which are called barcodes. During the continuous generation of droplets, the cells inside the droplets release large numbers of mRNAs due to the action of the lysate. At the same time, the mRNAs can be captured by the micro-particles. After all the droplets are generated, the droplets are broken. Then, the mRNAs are reversely transcribed into cDNAs and further amplified. Then, cDNAs are fragmented by treatment with a fragmentase, and sequencing adapters are added to both ends, afterwards, cDNAs with the adapters are sequenced. However, in this method, in order to minimize the probability of micro-particle doublets or cell doublets, it is necessary to reduce the density of the micro-particle phase and the cell phase to very low extents. Therefore, this method is difficult to achieve ultra-high throughput single-cell sequencing of more than 10000 cells per experiment, and the cost per cell increases invisibly.

Therefore, it is urgent to modify the traditional droplet microfluidic method and the cell pre-indexing method to improve the efficiency.

### SUMMARY

A main purpose of the present invention is to provide a construction method and a sequencing method for a single-cell transcriptome sequencing library, and a kit for preparing the single-cell transcriptome library, as to solve the problem of low efficiency in constructing the single-cell transcriptome sequencing library in the prior art.

In order to achieve the above purpose, according to a first aspect of the present invention, the present invention provides a construction method for a single-cell transcriptome sequencing library, and the method comprises the following steps:
1): performing in-situ reverse transcription in a cell or a cell nucleus on single-cell suspension to generate a full-length first chain cDNA with a first barcode; 2) overloading the cell or the cell nucleus obtained in Step 1) after the in-situ reverse transcription and a polymerase chain reaction (PCR) system used for second chain cDNA synthesis together with a magnetic bead capable of recognizing the first barcode into a droplet, and performing a droplet PCR; 3) recovering the magnetic bead after the droplet PCR; 4) performing a secondary amplification on cDNA on the magnetic bead obtained in Step 3); and 5) constructing a fragmentation library of cDNAs obtained in Step 4) after the secondary amplification to obtain the single-cell transcriptome sequencing library.

Further, in Step 1), an indexed oligo-dT primer is used for the in-situ reverse transcription to generate the full-length first chain cDNA with the first barcode; wherein, the oligo-dT primer sequentially comprises from 5′ to 3′: a. a magnetic bead capture region, which is a complementary region complementary to an oligonucleotide in a second barcode on the magnetic bead; b. a unique molecular identifier (UMI) used to recognize a single cDNA transcript; c. a first-round barcode used to distinguish different cells or cell nuclei; and d. a poly T sequence used to capture an mRNA; thus, the 5′ end of the obtained full-length first chain cDNA with the first barcode, sequentially comprises from 5′ to 3′: the magnetic bead capture region which is the complementary region complementary to the oligonucleotide in the second barcode on the magnetic bead; the unique molecular identifier; and the first-round barcode and the poly T sequence, wherein the magnetic bead is capable of recognizing the magnetic bead capture region in the first barcode.

Further, in Step 1), the in-situ reverse transcription is performed with a reverse transcriptase having terminal transferase activity, and the in-situ reverse transcription system further comprises a template switch oligo primer (Tn-TSO), preferably, the reverse transcriptase having terminal transferase activity is selected from Maxima H enzyme, SSII enzyme, or SSIV enzyme; and preferably, the template switch oligo primer is designed to comprise guanine located at the 3′ end, and preferably the number of the guanines is 3.

Further, the in-situ reverse transcription system further comprises glycerol, preferably, the final concentration of the glycerol in the in-situ reverse transcription system is 5%˜10%; preferably, the construction method further comprises performing immobilization and/or permeabilization pretreatment on the cell or the cell nucleus in the single-cell suspension before the in-situ reverse transcription; more preferably, an organic solvent is used for the immobilization and/or permeabilization pretreatment; further preferably, the organic solvent is methanol or paraformaldehyde; and furthermore preferably, the working concentration of the methanol is 50%˜100%, and the working concentration of the polyformaldehyde is 1%˜4%.

Further, in Step 2), the density of the cells or cell nuclei in the single-cell suspension is controlled to be 100˜1000 per μL, and the density of the magnetic beads is controlled to be 2000˜5000 per μL; and preferably, the density of the cells or cell nuclei is 500 per μL, and the density of the magnetic beads is 3000 per μL.

Further, in Step 2), the PCR system for second chain cDNA synthesis comprises: a primer simultaneously complementary to the 3′ end of the full-length first chain cDNA and a magnetic bead oligo primer, as well as a PCR reagent; and preferably, the primer simultaneously complementary to the 3′ end of the full-length first chain cDNA and the magnetic bead oligo primer is a Tn primer.

Further, in Step 2), the magnetic bead is a magnetic bead with a second barcode, preferably, the second barcode, according to a distance from the magnetic bead, sequentially comprises from near to far: a Tn primer, a second-round barcode, and a cDNA capture sequence for recognizing and capturing the full-length first chain cDNA; more preferably, the second-round barcode comprises a second-round first barcode and a second-round second barcode; further preferably, a first linker is further comprised between the second-round first barcode and the second-round second barcode; and furthermore preferably, a second linker is further comprised between the Tn primer and the magnetic bead.

Further, Step 5) comprises: constructing a fragmentation library of cDNAs obtained in Step 4) after secondary amplification to obtain a linear cDNA library, wherein the linear cDNA library is the single-cell transcriptome sequencing library; preferably, the linear cDNA library is further prepared into a circular cDNA library, wherein the circular cDNA library is the single-cell transcriptome sequencing library; preferably, the step of preparing the linear cDNA library into the circular cDNA library comprises: melting a double-stranded cDNA in the linear cDNA library under the action of a cyclization auxiliary sequence to obtain a melt product; ligating the melt product using a DNA ligase to obtain a single-stranded cyclization product; performing enzymatic digestion on the single-stranded cyclization product to degrade the remaining non-cyclized single-stranded cDNA and double-stranded cDNA to obtain the circular cDNA library; more preferably, after the enzymatic digestion of the single-stranded cyclization product, and further purifying the single-stranded cyclization product digested to obtain the circular cDNA library; and further preferably, wherein the single-stranded cyclization product is purified with a magnetic bead to obtain the circular cDNA library.

According to a second aspect of the present invention, the present invention provides a sequencing method for a single-cell transcriptome library, wherein the sequencing method comprises:

constructing a single-cell transcriptome sequencing library according to the construction method in the first aspect of the present invention; and performing sequencing by a sequencer on the single-cell transcriptome sequencing library.

According to a third aspect of the present invention, the present invention provides a kit for preparing a single-cell transcriptome library, wherein the kit comprises at least one of the following: an indexed oligo-dT primer with a first barcode, a template switch oligo primer, a reverse transcriptase, glycerol, an immobilization and/or permeabilization reagent, a primer simultaneously complementary to a 3′ end of a full-length first chain cDNA and a magnetic bead oligo primer, a magnetic bead with a second barcode, reagents for a linear cDNA library construction, and reagents for a circular cDNA library construction; preferably, the indexed oligo-dT primer sequentially comprises from 5′ to 3′: a. a magnetic bead capture region which is a complementary region complementary to an oligonucleotide in the second barcode on the magnetic bead; b. a unique molecular identifier used to recognize a single cDNA transcript; c. a first-round barcode used to distinguish different cells or cell nuclei; and d. a poly T sequence used to capture an mRNA; preferably, the reverse transcriptase is a reverse transcriptase having terminal transferase activity, and more preferably, the reverse transcriptase having terminal transferase activity is selected from Maxima H enzyme, SSII enzyme, or SSIV enzyme; preferably, the template switch oligo primer is designed to comprise guanine, and preferably the number of the guanines is 3; preferably, the final concentration of the glycerol in the in-situ reverse transcription system is 5%˜10%; preferably, the immobilization and/or permeabilization reagent is an organic solvent, more preferably, the organic solvent is methanol or paraformaldehyde, and furthermore preferably, the working concentration of the methanol is 50%˜100%, and the working concentration of the polyformaldehyde is 1%˜4%; preferably, the primer simultaneously complementary to the 3′ end of the full-length first chain cDNA and the magnetic bead oligo primer is a Tn primer; preferably, in the magnetic bead with the second barcode, the second barcode, according to a distance from the magnetic bead, sequentially comprises from near to far: a Tn primer, a second-round barcode, and a cDNA capture sequence for capturing the first chain cDNA; more preferably, the second-round barcode comprises a second-round first barcode and a second-round second barcode; further preferably, a first linker is further comprised between the second-round first barcode and the second-round second barcode; and furthermore preferably, a second linker is further comprised between the Tn primer and the magnetic bead; preferably, the reagents for a linear cDNA library construction are used for reverse transcription and fragmentation library construction of amplified cDNA to obtain a linear cDNA library; and preferably, the reagents for a circular cDNA library construction are used to prepare the linear cDNA library into a circular cDNA library.

By applying technical solutions of the present invention, the overloading of the cells or cell nuclei after the in-situ reverse transcription together with the pretreated magnetic bead is achieved, and an efficient, flexible, and easy-to-use large-scale scRNA sequencing method is provided. The method of the present invention is simple in operation, high in efficiency, and can efficiently construct the single-cell transcriptome sequencing library, thereby the throughput of the single-cell transcriptome sequencing method based on the droplet microfluidics is greatly improved, and the level of obtaining 10,0000 cell in a single experiment is achieved. In addition, the present invention saves the costs in experiments, and thus it is beneficial for the development of large-scale single-cell research projects.

### BRIEF DESCRIPTION OF THE DRAWINGS

Drawings of the present invention for constituting a part of the present application are used to provide further understanding of the present invention, and schematic embodiments of the present invention and descriptions thereof are used to explain the present invention and do not constitute improper limitations on the present invention. In the drawings:

FIG. 1 shows a flow diagram of a sci-RNA-seq technology in the prior art. In this technology, cells are first distributed evenly into a 96-well plate, and a type of indexed oligo-dT primer is added to each well. In other words, there are a total of 96 types of barcodes in the first-round indexing. Then the same reverse transcription reagent is added to each well for a reverse transcription reaction. After the first-round indexing, all cells or cell nuclei inside the well plate are collected. The collected product is distributed evenly into a second 96-well plate, and similarly, a different type of indexed primer is added to each well. There are 96 or 384 barcodes in the second-round indexing in total. The second round performs indexing by PCR.

FIG. 2 shows a flow diagram of a single-cell sequencing technology based on droplet microfluidics in the prior art. In this technology, a water-in-oil droplet is first used to encapsulate a cell and a micro-particle to obtain a droplet simultaneously containing one cell and one micro-particle, and other components of the droplet also include a lysate, and the micro-particle comprises a large number of DNA sequences with unique molecular identifiers, which are called barcodes. During the continuous generation of droplets, the cells inside the droplets are lysed by the lysate, and a large amount of mRNA is released. At the same time, mRNA can be captured by the micro-particles. After all the droplets are generated, the droplets are broken. Afterwards, mRNA is reversely transcribed into cDNA and further amplified. Then, the cDNA is fragmented by a fragmentase, and sequencing adapters are added to both ends, afterwards cDNA with the adapters is sequenced.

FIG. 3 shows a flow diagram of a construction method for a single-cell transcriptome sequencing library and a sequencing method for a single-cell transcriptome library according to the present invention. Firstly, single-cell suspension is subjected to in-situ reverse transcription in the cell or cell nucleus to generate a full-length first chain cDNA with a first barcode; then, the obtained cells or cell nuclei after the in-situ reverse transcription and a PCR system used for second chain cDNA synthesis are overloaded together with a magnetic bead capable of recognizing the first barcode into a droplet, and a droplet PCR is performed; then, the magnetic beads after the droplet PCR are recovered; secondary amplification is performed on cDNA on the magnetic beads; and a fragmentation library of cDNA after the secondary amplification is constructed to obtain the single-cell transcriptome sequencing library. Sequencing is performed by a sequencer on the single-cell transcriptome sequencing library obtained.

FIG. 4 shows a droplet generation operation for achieving overloading according to an embodiment of the present invention. A protective film on the surface of a chip is tore off, and the chip is placed in a chip slot area of a droplet generation apparatus; an end A of a connecting tube on a collection cover (it is a connecting tube in contact with the bottom of a collection tube) is inserted into an outlet hole of the chip; a 50 mL syringe (BD, 300136) is placed on a fixing bracket, and a push rod is adjusted to an initial position. The syringe is connected to an end B of the connecting tube on the collection tube cover (it is a connecting tube that is not in contact with the bottom of the collection tube) with a flat-port needle. Subsequently, 200 μL of droplet generation oil is added to the collection tube, the collection cover is tightened, and the collection tube is vertically placed on the fixing bracket. Suspension of the cells or cell nuclei is evenly mixed by gently blowing and sucking with a pipette, then 100 μL of the suspension of the cells or cell nuclei is added to the cell well of the chip while ensuring that the pipette tip contacts the bottom of the well. The magnetic beads are evenly mixed by gently blowing and sucking with the pipette, and 100 μL of the magnetic beads evenly mixed by blowing and sucking is added to the magnetic bead well of the chip while ensuring that the pipette tip contacts the bottom of the well. 350 μL of the droplet generation oil is immediately added to the oil well of the chip, the push rod of the syringe is quickly pulled to a slot position and is clamped in the slot, and a timer is started. After 5 min, the collection of the droplets is started, the collection cover on the collection tube is immediately loosened, the connecting tube in the chip outlet hole is pulled out, and the connecting tube is vertically stretched, so that droplets in the tube are flowed into the collection tube, then the collection cover is replaced with a regular collection tube cover.

FIG. 5 shows a result of ultra-high throughput single-cell transcriptome sequencing using a human-derived 293T cell line according to an embodiment of the present invention. In this figure, the used sample is the human-derived 293T cell line fixed with methanol, 24 types of barcodes are used for the first-round indexing in this experiment, and finally, nearly 20000 usable cells are obtained with a single chip.

FIG. 6 shows a result of ultra-high throughput single-cell transcriptome sequencing using a mixed cell line of human-derived 293T and mouse-derived 3T3 according to an embodiment of the present invention. In this figure, the used sample is the mixed cell line of human-derived 293T and mouse-derived 3T3 fixed with methanol, 48 barcodes are used for the first-round indexing in this experiment, and finally, about 30000 usable cells are obtained with a single chip. The doublet rate of human and mouse cells is 1.59%.

FIG. 7 shows a result of ultra-high throughput single-cell transcriptome sequencing using a mixed cell line of human-derived 293T and mouse-derived 3T3 according to an embodiment of the present invention. In this figure, the used sample is the mixed cell line of human-derived 293T and mouse-derived 3T3 fixed with methanol, 96 types of barcodes are used for the first-round indexing in this experiment, and finally, 80000 usable cells are obtained with a single chip. The doublet rate of human and mouse cells is 10.65%.

### DETAILED DESCRIPTION OF THE EMBODIMENTS

It should be noted that, in the case without conflicting, embodiments in the present application and features in the embodiments could be combined with each other. The present invention is described in detail below with reference to the drawings and in combination with the embodiments.

As described in the background section, the conventional methods based on a large number of cells for analyzing the molecular contents of biological samples mask the heterogeneity of the cells in biological systems. Therefore, more and more research has been performed at the single-cell level, and the analysis of the transcriptome of a single cell has already become an effective strategy to solve the biological heterogeneity. Although methods of single-cell RNA sequencing (sc-RNA-seq) have rapidly increased, traditional methods still have many problems.

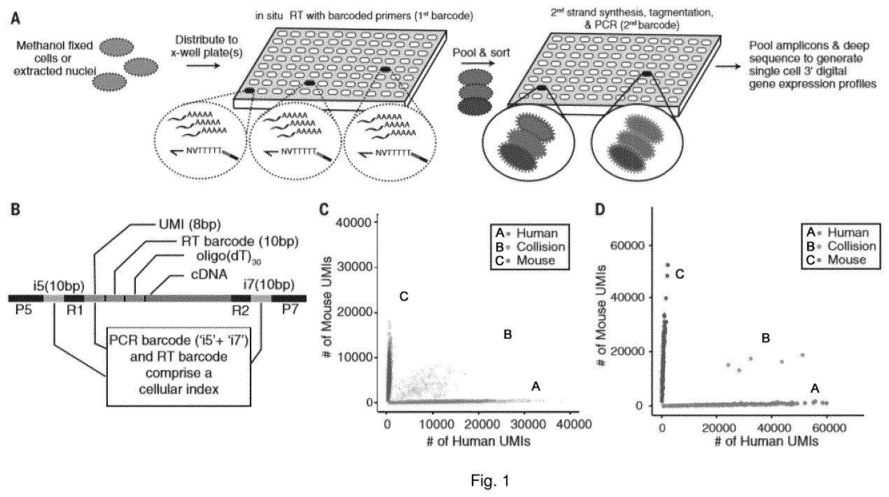

FIG. 1 shows a flow diagram of an sci-RNA-seq technology in the prior art. In this traditional multi-round cell indexing method, although tens of thousands of cells can be obtained in a single experiment, which greatly improves the throughput of a single experiment and saves costs, the disadvantage of this experimental method is that it is a manual operation based on a well plate, which cannot achieve automated cell capture and greatly increases the workload; in addition, its experimental steps are very cumbersome; and each round of cell recovery causes a certain amount of loss, resulting in a relatively low cell recovery rate.

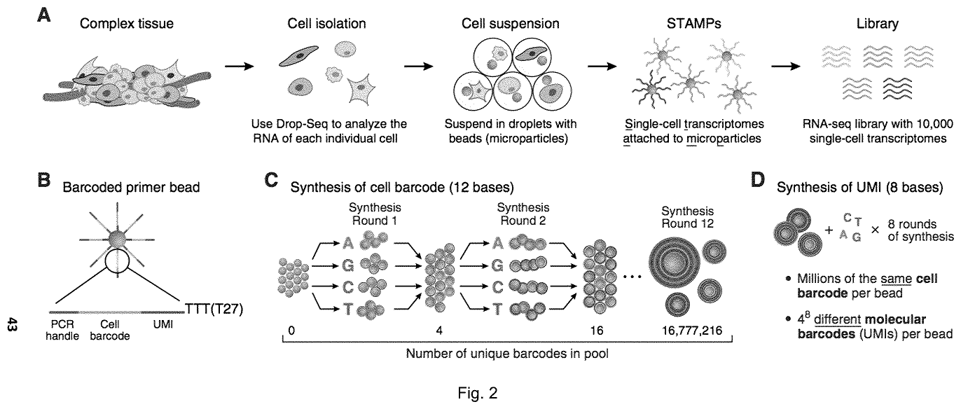

FIG. 2 shows a flow diagram of a single-cell sequencing technology based on droplet microfluidics in the prior art. In this traditional single-cell sequencing technology based on the droplet microfluidics, in order to minimize the probability of micro-particle doublets or cell doublets, it is necessary to reduce the density of the micro-particle phase and cell phase to a very low extent. Therefore, this method is difficult to achieve ultra-high throughput single-cell sequencing of more than 10000 cells per experiment, and the cost per cell increases invisibly.

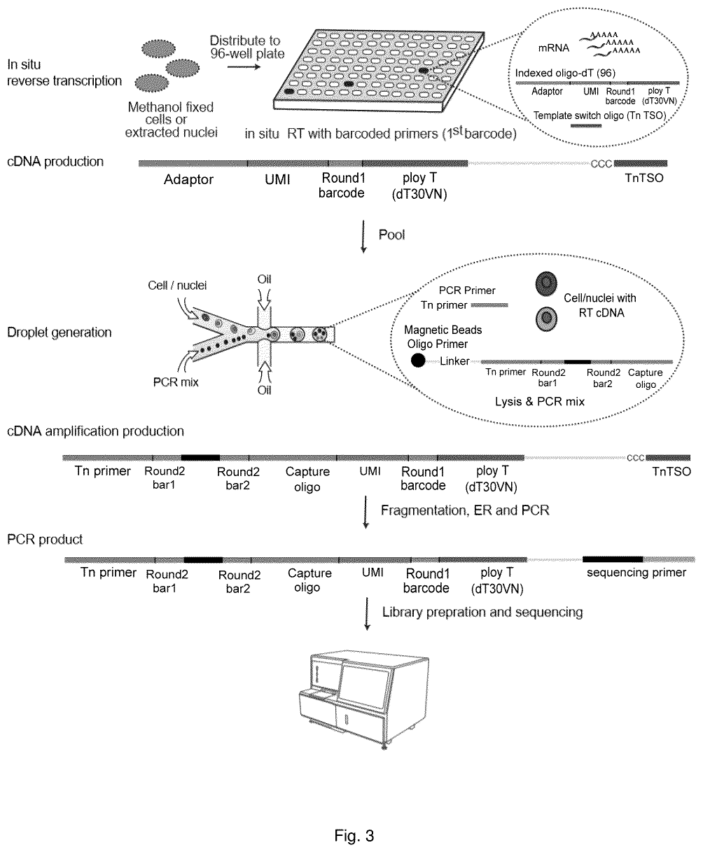

FIG. 3 shows a flow diagram of a construction method for a single-cell transcriptome sequencing library and a sequencing method for a single-cell transcriptome library according to the present invention. In order to modify a traditional droplet microfluidic method and a cell pre-indexing method, the present invention provides a construction method for a single-cell transcriptome sequencing library and a sequencing method for a single-cell transcriptome library based on this construction method for the single-cell transcriptome sequencing library.

In the construction method for the single-cell transcriptome sequencing library according to an embodiment of the present invention, in-situ reverse transcription in a cell or a cell nucleus is first performed on single-cell suspension in a well plate to generate a full-length first chain cDNA with a first barcode, thereby the cell or cell nucleus is indexed in advance by the in-situ reverse transcription. In this step, the number of types of barcodes for all cells ranges from 24 to 384. Subsequently, the cell or cell nucleus after the in-situ reverse transcription and a PCR system used for second chain cDNA synthesis are overloaded together with a magnetic bead into a droplet. Usually, the droplet has 1˜5 cells or cell nuclei, and correspondingly, it is ensured that there are 1˜10 magnetic beads in the droplet. By overloading, almost all droplets contain magnetic beads and the encapsulation rate of viable cells or cell nuclei is also greatly improved. The PCR system used for the second chain cDNA synthesis is loaded while the cell or cell nucleus is overloaded with the magnetic bead, and a droplet PCR is performed, so that the magnetic beads in the droplet can uniformly capture cDNAs obtained by the in-situ reverse transcription. Afterwards, cDNAs are subjected to secondary amplification and a cDNA library is constructed.

In the sequencing method for the single-cell transcriptome library according to an embodiment of the present invention, a single-cell transcriptome sequencing library is first constructed, and then sequencing is performed by a sequencer on the single-cell transcriptome sequencing library. After high-throughput sequencing data is obtained, the magnetic beads from the same droplet are first combined according to the correlation of fragments captured by the magnetic beads. In one droplet, cells are lysed and cDNAs are released, and the magnetic beads in the droplet randomly capture cDNAs released. Therefore, the fragments captured by the magnetic beads in one droplet are all cDNA fragments of the cells in this droplet. Meanwhile, a cDNA template is amplified by PCR in the droplet. After the amplification is completed, the copy number of the template increases, and the probability of each replicated template being captured by the different magnetic beads in the same droplet is equal. Therefore, even if there is a plurality of different magnetic beads in the same droplet, the fragments captured by them should be very consistent. Therefore, this type of consistent magnetic beads can be combined by calculating the correlation of the fragments captured by the magnetic beads. Therefore, according to the method of the present invention, the overloading of the magnetic beads in the droplet is achieved, as to improve the utilization efficiency of the droplet. Subsequently, according to a first-round barcode, the overloaded cells or cell nuclei in the same droplet are distinguished, thereby the doublet situation is distinguished while the cell density increases. In the present invention, the doublet refers to the presence of two or more cells in one droplet; and if the doublet cannot be distinguished, only the sum of the transcripts from these two cells can be obtained from sequencing data, and the characteristics of a single cell cannot be obtained. It may cause difficulties in downstream analysis of single cells. Therefore, it is necessary to distinguish the doublet situation.

During the calculation of the correlation, the magnetic beads are distinguished according to a pre-indexed cell barcode. At this time, one cell is divided into a plurality of sub-cells, and they are combined according to the correlation of unique molecule identifiers (UMI) captured by the different sub-cells. Since PCR is performed on the unique molecule identifier in the droplet, there are multiple copies of the unique molecule identifier of the same cell, then the multiple copies are captured by the different magnetic beads in the droplet. If the plurality of the sub-cells has more identical unique molecule identifiers, it is indicated that they capture the same cell.

Subsequently, according to the correlation of the unique molecule identifiers, the magnetic beads in one droplet are distinguished, and the consistent magnetic beads are combined. Firstly, a matrix of sub-cells * UMI is created, the value of the unique molecule identifier number 1 is replaced with 0 (in this case, UMI=1 is used as the background), a Jaccard index between the sub-cells is calculated, and a combination threshold is selected according to a knee plot of the Jaccard index. If the Jaccard index among the plurality of the sub-cells is higher than this threshold, they are combined into one cell.

The overloading of the magnetic beads in the droplet is achieved by increasing the density of the magnetic bead phase. According to a probability density calculation formula of Poisson distribution, in the case that the droplet flow rate is consistent in this apparatus, when the droplet size is 110 μm, if the density of particles reaches 3000/μL, about 70% of the droplets could encapsulate the particle. The density of the magnetic beads used in this experiment is 3000/μL. After calculation, the probability of encapsulating one magnetic bead in the droplet is 36.7%, and the probability of encapsulating two or more magnetic beads is 30%. When the probability of two or more magnetic beads being encapsulated by the droplet is greater than 5%, it is considered that the overloading of the magnetic beads in the droplet is achieved. The probability density function of the Poisson distribution used here is as follows:

$$Poisson ( N ( t ) = n ) = ( {( λ t )}^{n} {e}^{- λ t} ) / n ! , ( λ t > 0 , n \geq 0 ) ,$$

wherein 2 is an average number of cells in each droplet, and t is a constant.

When overloaded cells are distinguished according to the first-round barcode, the oligo-dT used in the first round carries a total of 96 to 384 types of barcodes, it is equivalent that the cells are indexed into 96 to 384 types in the first round. In the process of generating the droplets that encapsulate the cells, according to the Poisson distribution calculation formula, the cell density is artificially controlled, so that there are no more than 10 cells in each droplet. It is equivalent that 10 cells are randomly selected from 96 to 384 types of the cells, there is a high probability that these 10 cells could carry 10 different barcodes in the first round. At the same time, the magnetic beads in each droplet carry different barcodes as second-round barcodes. After sequencing is completed, the cells from the same droplet can be distinguished according to indexing sequences of the two rounds.

In an embodiment, the construction method for the single-cell transcriptome sequencing library comprises the following steps:
1) performing in-situ reverse transcription in a cell or a cell nucleus on single-cell suspension to generate a full-length first chain cDNA with a first barcode;
2) overloading the cell or the cell nucleus obtained in Step 1) after the in-situ reverse transcription and a PCR system used for second chain cDNA synthesis together with a magnetic bead capable of recognizing the first barcode into a droplet, and performing a droplet PCR;
3) recovering the magnetic bead after the droplet PCR;
4) performing secondary amplification on cDNAs on the magnetic bead obtained in Step 3); and
5) constructing a fragmentation library of cDNA obtained in Step 4) after the secondary amplification to obtain the single-cell transcriptome sequencing library.

In an embodiment, in Step 1), an indexed oligo-dT primer containing a barcode is used for the in-situ reverse transcription to generate the full-length first chain cDNA with the first barcode. The oligo-dT primer sequentially comprises from 5′ to 3′:
a. a magnetic bead capture region which is a complementary region (also known as an adaptor) complementary to an oligonucleotide in a second barcode on the magnetic bead and is used for the magnetic bead capturing cDNAs;
b. a unique molecular identifier (UMI) used to recognize a single cDNA transcript;
c. a first-round barcode (Round 1 Barcode) used to distinguish different cells or cell nuclei by indexing the cells; and
d. a poly T sequence (dT30VN) used to capture mRNA.

Thus, by using the oligo-dT primer, the 5′ end of the obtained cDNA product, sequentially comprises from 5′ to 3′: the magnetic bead capture region which is the complementary region (also known as the adaptor) complementary to the oligonucleotide in the second barcode on the magnetic bead; UMI; the first-round barcode; and the poly T sequence (dT30VN). The magnetic bead is capable of recognizing the magnetic bead capture region in the first barcode.

The in-situ reverse transcription is performed with a reverse transcriptase having terminal transferase activity. Preferably, the reverse transcriptase having terminal transferase activity is selected from a Maxima H enzyme, SSII enzyme, or SSIV enzyme. By the action of the reverse transcriptase, a cytosine (C) is added to a tail end of the cDNA first chain, and preferably, the number of the cytosines is 3. At this time, the cDNA first chain is obtained, and its structure can be represented as: magnetic bead capture region—UMI—Round 1 Barcode—poly T sequence—cDNA-CCC.

In an embodiment, the in-situ reverse transcription system further comprises a template switch oligo primer (also known as a template switch oligo (Tn-TSO)). In an embodiment, the sequence of the template switch oligo primer is: 5′-CGTAGCCATGTCGTTCTGrGrG+G-3′, wherein “+G” represents a guanine with locked nucleotide modification, and “rG” is a guanine nucleotide. Therefore, the template switch oligo primer is designed to comprise guanine nucleotide (G) located at the 3′-end, and preferably the number of the guanine nucleotides is 3 (Tn-TSO-rGrG+G). In this way, the template switch oligo primer with the guanine forms complementary pairing with the cytosine on cDNA, namely, 3′-end CCC of the obtained cDNA product (cDNA first chain) is complementary to the 3′-end rGrG+G of the Tn-TSO-rGrG+G, allowing the template switch oligo primer to sequentially serve as a reverse transcription template for guiding the cDNA synthesis.

In an embodiment, the in-situ reverse transcription system further comprises glycerol, preferably, the final concentration of the glycerol in the in-situ reverse transcription system is 5%˜10%. The glycerol could protect the integrity of the cells or cell nuclei. In addition, the glycerol could reduce intercellular adhesion and indirectly improve the efficiency of the reverse transcription.

In an embodiment, pretreatment of the cells or cell nuclei could be performed before the in-situ reverse transcription. The pretreatment comprises immobilization and permeabilization with an organic solvent. Preferably, the organic solvent is, for example, methanol and paraformaldehyde. In an embodiment, the working concentration of the methanol is 50%˜100%, and the working concentration of the paraformaldehyde is 1%˜4%. The immobilization could reduce the free degree and loss amount of mRNA during the reverse transcription. The permeabilization could improve the permeability of the cells or cell nuclei. Both the immobilization and permeabilization could improve the efficiency of the reverse transcription and template switch while ensuring the integrity of the cells or cell nuclei.

By pretreating cells or cell nuclei and comprising glycerol in an in-situ reverse transcription system, it is also possible to simultaneously encapsulate multiple cells in one droplet in a subsequent operation, thereby the capture efficiency of the droplet is greatly improved.

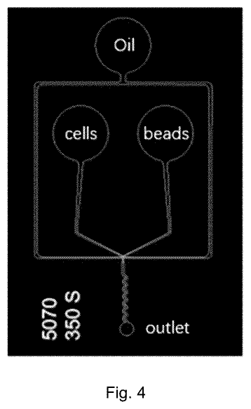

In an embodiment, the method of the present invention comprises overloading the cells or cell nuclei together with pre-treated magnetic beads after the in-situ reverse transcription. FIG. 4 shows a droplet generation operation for achieving overloading according to an embodiment of the present invention. By controlling the density of the cells or cell nuclei in the single-cell suspension to 100˜1000 per μL and controlling the density of the magnetic beads to 2000˜5000 per μL, the overloading is achieved. In a preferred embodiment, the density of the cells or cell nuclei is 500 per μL and the density of the magnetic beads is 3000 per μL. By adjusting the concentration of the cells or cell nuclei in this step, it is suitable for a subsequent in-situ reverse transcription operation and the efficiency of the in-situ reverse transcription is improved. By simultaneously encapsulating multiple magnetic beads in one droplet, the utilization efficiency of the magnetic beads is improved.

In an embodiment, the method of the present invention comprises performing a droplet PCR. A PCR system for second chain cDNA synthesis is added to the droplet PCR. In an embodiment, the PCR system for the second chain cDNA synthesis comprises: a primer simultaneously complementary to the 3′ end of the full-length first chain cDNA and a magnetic bead oligo primer, as well as a PCR reagent. Preferably, the primer simultaneously complementary to the 3′ end of the full-length first chain cDNA and the magnetic bead oligo primer is a Tn primer. In an embodiment, the sequence of the Tn primer is 5′-CGTAGCCATGTCGTTCTG-3′. The Tn primer is consistent with the 5′ end of the magnetic bead sequence and is complementary to the 3′ end of the cDNA template, which is capable of achieving exponential amplification of cDNA in the droplet.

In an embodiment, the magnetic bead is a magnetic bead with a second barcode, and the second barcode, according to a distance from the magnetic bead, sequentially comprises from near to far: a Tn primer, a second-round barcode (Round2 Barcode), and a cDNA capture sequence (Capture oligo), and the cDNA capture sequence captures the first chain cDNA by recognizing the first barcode on the full-length first chain cDNA with the first barcode generated by the in-situ reverse transcription. In an embodiment, the capture sequence in the second barcode of the magnetic bead has the following sequence: 5′-TCGTCGGGCAGCGTC-3′. In an embodiment, the second-round barcode comprises a second-round first barcode (Round2 Bar1) and a second-round second barcode (Round2 Bar2). In an embodiment, the second-round barcode comprises a second-round first barcode, a second-round second barcode, and a first linker (linkage sequence) between them. In an embodiment, the sequence of the first linker is: 5′-CCTTCC-3′. In the preparation process of the magnetic bead, the first linker is designed to link the second-round first barcode and the second-round second barcode, and it does not participate in capturing cDNA. In an embodiment, a second linker is further comprised between the Tn primer and the magnetic bead. The second linker is 5 T bases labelled with biotin at the 5′ end, and the sequence is: 5′-biotin-TTTTT-3′. The biotin can specifically bind to streptavidin on the magnetic bead, so the second linker can link the oligonucleotide to the magnetic bead. PCR amplification of cDNA is completed in the droplet by the droplet PCR, so that the multiple magnetic beads in the same droplet fully capture cDNAs. Since each cDNA molecule carries different UMI, the multiple magnetic beads in the same droplet can be combined according to the correlation of UMI captured by them, so that the magnetic beads from the same droplet can be distinguished.

In an embodiment, the method of the present invention further constructs a fragmentation library of cDNA obtained in Step 4) after the secondary amplification. By constructing the fragmentation library, a linear cDNA library is obtained, and the linear cDNA library is the single-cell transcriptome sequencing library. In an embodiment, the linear cDNA library is further prepared into a circular cDNA library, and the circular cDNA library is the single-cell transcriptome sequencing library.

In an embodiment, the step of preparing the linear cDNA library into the circular cDNA library comprises: melting a double-stranded cDNA in the linear cDNA library under the action of a cyclization auxiliary sequence to obtain a melt product; ligating the melt product using a DNA ligase to obtain a single-stranded cyclization product; performing enzymatic digestion on the single-stranded cyclization product to degrade the remaining non-cyclized single-stranded cDNA and double-stranded cDNA to obtain the circular cDNA library.

In an embodiment, after the enzymatic digestion of the single-stranded cyclization product, the single-stranded cyclization product digested is further purified to obtain the circular cDNA library. In an embodiment, the single-stranded cyclization product is purified with a magnetic bead to obtain the circular cDNA library.

The sequencing method for the single-cell transcriptome library according to an embodiment of the present invention first constructs the single-cell transcriptome sequencing library as described above, and then performs sequencing by a sequencer on the single-cell transcriptome sequencing library.

A kit for a single-cell transcriptome sequencing according to an embodiment of the present invention comprises at least one of the following: an indexed oligo-dT primer with a first barcode, a template switch oligo primer, a reverse transcriptase, glycerol, an immobilization reagent and/or permeabilization reagent, a primer simultaneously complementary to the 3′ end of a first chain cDNA and a magnetic bead oligo primer, a magnetic bead with a second barcode, reagents for a linear cDNA library construction, and reagents for a circular cDNA library construction.

In an embodiment, the oligo-dT primer sequentially comprises from 5′ to 3′: a. a magnetic bead capture region which is a complementary region complementary to an oligonucleotide in the second barcode on the magnetic bead; b. a unique molecular identifier used to recognize a single cDNA transcript; c. a first-round barcode used to distinguish different cells or cell nuclei; and d. a poly T sequence used to capture an mRNA. Thus, the 5′ end of the obtained cDNA product sequentially comprises from 5′ to 3′: the magnetic bead capture region which is the complementary region complementary to the oligonucleotide in the second barcode on the magnetic bead; the unique molecular identifier; the first-round barcode and the poly T sequence.

In an embodiment, the template switch oligo primer is designed to contain guanine, and preferably, the number of the guanines is 3. In an embodiment, the reverse transcriptase is a reverse transcriptase having terminal transferase activity, and more preferably, the reverse transcriptase having terminal transferase activity is selected from Maxima H Minus transcriptase (Thermo Fisher, EP0752), SuperScript IV transcriptase (Thermo Fisher, EP0752, 18090010), or SuperScript™ II transcriptase (Thermo Fisher, 18064022). In an embodiment, the final concentration of the glycerol in the in-situ reverse transcription system is 1%˜10%. In an embodiment, the reagent is methanol or paraformaldehyde. In an embodiment, the working concentration of the methanol is 30%˜100%, and the working concentration of the polyformaldehyde is 0.1%˜4%.

In an embodiment, in the magnetic bead with the second barcode, the second barcode, according to a distance from the magnetic bead, sequentially comprises from near to far: a Tn primer, a second-round barcode, and a cDNA capture sequence for capturing the first chain cDNA; more preferably, the second-round barcode comprises a second-round first barcode and a second-round second barcode; further preferably, a first linker is further comprised between the second-round first barcode and the second-round second barcode; and furthermore preferably, a second linker is further comprised between the Tn primer and the magnetic bead.

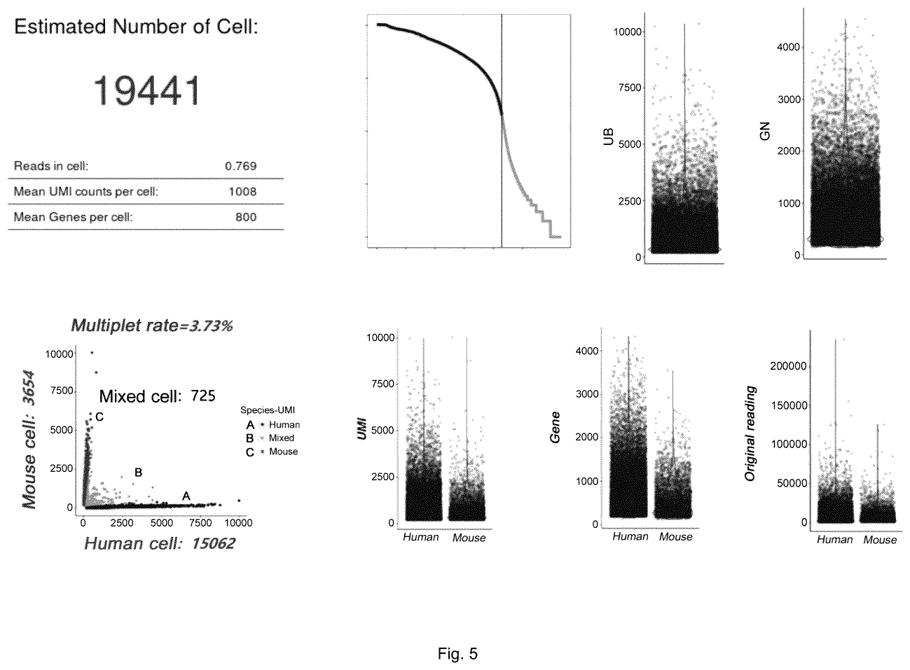

FIG. 5 shows a result of ultra-high-throughput single-cell transcriptome sequencing using a human-derived 293T cell line according to an embodiment of the present invention. In this figure, the used sample is the human-derived 293T cell line fixed with methanol, 24 types of barcodes are used for the first-round indexing in this experiment, and finally, nearly 20000 usable cells are obtained with a single chip.

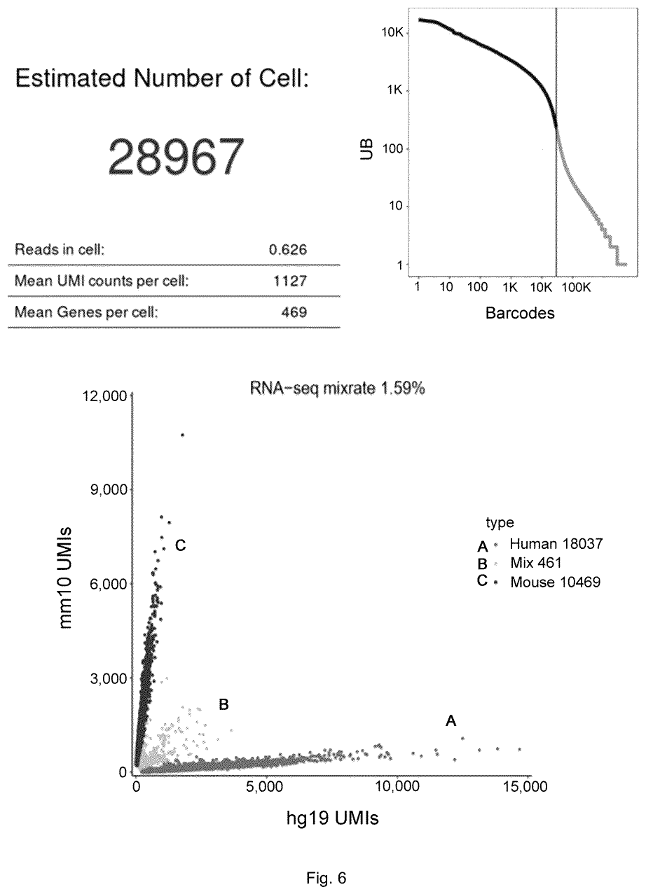

FIG. 6 shows a result of ultra-high-throughput single-cell transcriptome sequencing using a mixed cell line of human-derived 293T and mouse-derived 3T3 according to an embodiment of the present invention. In this figure, the used sample is the mixed cell line of human-derived 293T and mouse-derived 3T3 fixed with methanol, 48 types of barcodes are used for the first-round indexing in this experiment, and finally, about 30000 usable cells are obtained with a single chip. The doublet rate of human and mouse cells is 1.59%.

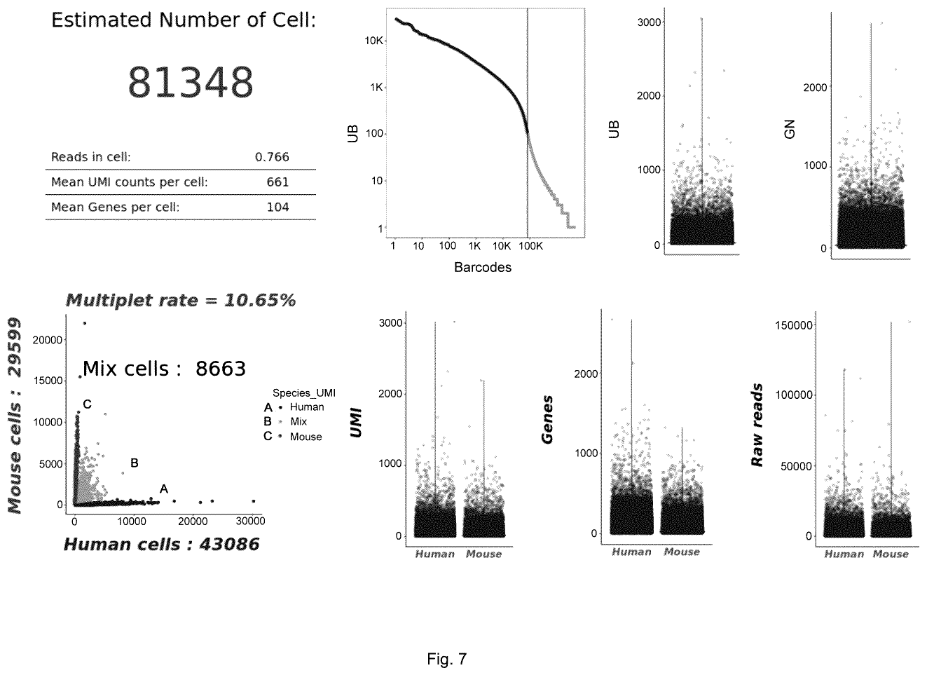

FIG. 7 shows a result of ultra-high-throughput single-cell transcriptome sequencing using a mixed cell line of human-derived 293T and mouse-derived 3T3 according to an embodiment of the present invention. In this figure, the used sample is the mixed cell line of human-derived 293T and mouse-derived 3T3 fixed with methanol, 96 types of barcodes are used for the first-round indexing in this experiment, and finally, 80000 usable cells are obtained with a single chip. The doublet rate of human and mouse cells is 10.65%.

The present invention is further described in detail below in combination with specific embodiments, and these embodiments could not be understood as limiting the scope of protection claimed by the present invention.

### Example 1: Material and Method

### Preparation of Single-Cell Suspension

Preparation steps for single-cell suspension were performed before in-situ reverse transcription.

The suspension of single cells or cell nuclei was prepared from obtained cell lines or solid tissues using an appropriate digestion method and/or grinding method, the prepared suspension of the single cells or cell nuclei was washed with phosphate buffer solution (PBS) containing 0.04% bovine serum albumin (BSA) 1-2 times, and a 40 um cell sieve (BD, 352340) was used for filtration. Subsequently, the single cells or cell nuclei were fixed, for example, cold methanol might be used for immobilization for 20 min. The fixed single cells or cell nuclei were washed with PBS containing 0.04% BSA for times, and a 40 um cell sieve was used for filtration. Subsequently, a precipitate of the single cells or cell nuclei was collected after centrifugation at 300-500 g and 4° C. for 5 min. Afterwards, 100 μL of a cell resuspension buffer was added to resuspend the cells or cell nuclei, and the concentration of the cells or cell nuclei was detected and adjusted to be 5000/μL using a cell counting plate or a counter.

### In-Situ Reverse Transcription in Cells or Cell Nuclei

Before in-situ reverse transcription, 25 μM indexed oligo-dT primer was prepared in a multi-well plate at 1 μL per well, and the multi-well plate was stored at −20° C. The sequence of the indexed oligo-dT primer was: 5′-TCGTCGGCAGCGTCAGATGTGTATAAGAGACNNNNNNNNNNNNNJJJJJJJJJJVTTTTTT TTTTTTTTTTTTTTTTTTTTTTTTVN-3′. Wherein, N represented any one of the 4 bases, of which the total is 13, and this segment was UMI; J was 96-384 types of designed barcodes, with a total of 10 bases, followed by 30 T; V represented any one of the 3 bases other than T; and the last one was a random base N.

At the beginning of the experiment, suspension of the single cells or cell nuclei was added at an amount of 10000 cells or cell nuclei/well (namely 2 μL suspension/well). Subsequently, the system was incubated at 55° C. for 5 min to unfold RNA secondary structure, and it was immediately placed on ice to prevent its recombination.

16 μL of reverse transcription mixed solution was added to each well, and the reverse transcription mixed solution comprised: 1×reverse transcription buffer, 0.5 mM dNTP, 2U/μL RNA enzyme inhibitor, 10U/μL Maxima H enzyme (Thermo Scientific, EP0751), 10% glycerol and 2.5 μM Tn-TSO (5′-CGTAGCCATGTCGTTCTGrGrG+G-3′), wherein “+G” represented a guanine with locked nucleotide modification, and “rG” was a guanine nucleotide).

A heat cover was set to 65° C. and the reverse transcription was performed according to the following procedure:
heating at 50° C. for 10 min;
performing the following steps for 3 cycles:
{maintaining at 8° C. for 12 s,
maintaining at 15° C. for 45 s,
maintaining at 20° C. for 45 s,
maintaining at 30° C. for 30 s,
maintaining at 42° C. for 2 min, and
maintaining at 50° C. for 3 min;}
heating at 50° C. for 15 min; and
storing at 4° C.

### Recovery of Cells or Cell Nuclei

The cells or cell nuclei after the in-situ reverse transcription were recovered. 40 μL of a washing buffer was added to each well, the washing buffer was PBS containing 0.04% BSA, and then the treated cells or cell nuclei were recovered. The resulting liquid after several times of washing was combined in a 1.5 mL centrifuge tube (Invitrogen, AM12400) to achieve the maximum recovery rate. The cells or cell nuclei were collected by centrifugation at 500 rcf and 4° C. for 10 min, and it was repeatedly washed with 3 mL of a washing buffer for 1 time. A precipitate obtained was resuspended in 50 μL of the washing buffer. 2 μL was taken and diluted for counting.

Then, 50000 cells were added to a PCR pre-mixed solution to reach a total volume of 100 μL for standby. The PCR pre-mixed solution used herein comprised: 20 μL of 1×Fidelity buffer (KAPABIOSYSTEM, KK2103), 3 μL of 0.3 mM dNTP mixture, 7 μL of 3.75 mM MgCl₂, 0.2 μM Tn primer, 16.7 μL of 10% Opti-prep (Sigma, D1556-250ML), and 8 μL of 1U/μL KAPA polymerase (added separately when running droplets); and finally, the total volume was replenished to 100 μL with H₂O. Since the density of Opti-prep was relatively high, the magnetic beads suspended in it might not affect the density due to sedimentation, so it might be used to suspend the magnetic beads.

### Pretreatment of Magnetic Beads

For each experiment, pipetting 100 μL of indexed (the index sequence was: 5′-TTTTTCCCGTAGCCATGTCGTTCTGCGJJJJJJJJJJCCTTCCJJJJJJJJJJTCGTCGGCAGCGTC-3′, wherein J bases made up a magnetic bead barcode sequence, there were a total of 1536 types, and two segments respectively, and after being randomly combined, there were a total of 1536*1536 types of the magnetic beads) magnetic beads (300,000) into a 0.2 mL PCR tube (Axygen, 14-222-260), placing it stilly on a magnetic rack for 2 min, and discarding the supernatant. Removing the PCR tube from the magnetic rack, adding 200 μL of 1×buffer D to suspend the magnetic beads, and incubating at a room temperature for 5 min. Wherein the buffer D comprised: 1 mM ethylene diamine tetraacetic acid (EDTA) and 9 mg/mL 85% KOH.

The PCR tube was stilly placed on a magnetic rack for 2 min, then the supernatant was discarded. The PCR tube was kept on the magnetic rack, 200 μL of 1×buffer D was added, and the supernatant was discarded after being stilly placed for 30 s. 200 μL of LSWB buffer was added, and then a supernatant was discarded after being stilly placed for 30 s. The previous operation was repeated. Wherein the LSWB buffer comprised: 50 mM Tris-HCl, 150 mM NaCl, 0.05% Tween-20, and H₂O.

Then 100 μL of a PCR pre-mixed solution was added to resuspend the magnetic beads for standby. The PCR pre-mixed solution used herein comprised: 40 μL of 0.02% SDS, 20 μL of 1×Fidelity buffer, 3 μL of 0.3 mM dNTP mixture, 7 μL of 3.75 mM MgCl2, 0.2 μM Tn primer, 16.7 μL of 10% Opti-prep, and 11.3 μL of H₂O.

### Generation of Droplets

Droplets needed to be generated before the droplet PCR. A protective film on the surface of a chip (Manufacturer: Hanguang; and Model: Dow Corning Type-184, 110-1) was tore off, and it was placed in a chip slot area of a droplet generation apparatus; an end A of a connecting tube on a collection cover (it was a connecting tube in contact with the bottom of the collection tube) was inserted into an outlet hole of the chip; a 50 mL syringe (BD, 300136) was placed on a fixing bracket, and a push rod was adjusted to an initial position. The syringe was connected to an end B of the connecting tube (it was a connecting tube that was not in contact with the bottom of the collection tube) on the collection tube cover with a flat-port needle. Subsequently, 200 μL of droplet generation oil (BIO-RAD, 1863005) was added to the collection tube, the collection cover was tightened, and the collection tube was vertically placed on the fixing bracket.

Suspension of cells or cell nuclei was evenly mixed by gently blowing and sucking with a pipette, then 100 μL of the suspension of the cells or cell nuclei was added to a cell well of the chip while ensuring that a pipette tip contacted the bottom of the well. The magnetic beads were evenly mixed by gently blowing and sucking with the pipette, 100 μL of the magnetic beads evenly mixed by blowing and sucking were added to the magnetic bead well of the chip while ensuring that the pipette tip contacted the bottom of the well. 350 μL of the droplet generation oil was immediately added to the oil well of the chip, the push rod of the syringe was quickly pulled to a slot position and was clamped in the slot, and a timer was started. After 5 min, the collection of the droplets was started, the collection cover on the collection tube was immediately loosened, the connecting tube in the chip outlet hole was pulled out, and the connecting tube was vertically stretched, so that the droplets in the tube were flowed into the collection tube, then the collection cover was replaced with a regular collection tube cover.

As described above, it was achieved by increasing the density of the magnetic bead phase. According to a calculation formula of Poisson distribution, in the case that the droplet flow rate was consistent in this apparatus, when the droplet size was 110 μm, if the density of particles reached 3000/μL, about 70% of the droplets might encapsulate the particles. The density of the magnetic beads used in this experiment was 3000/μL. After calculation, the probability of encapsulating 1 magnetic bead in the droplet was 36.7%, and the probability of encapsulating two or more magnetic beads was 30%. When the probability of two or more magnetic beads being encapsulated by the droplet was greater than 5%, it was considered that the overloading of the magnetic beads in the droplet was achieved.

### Droplet PCR

The droplets were transferred to an 8-tube PCR strip (Axygen, PCR-0208-C), it was ensured that the liquid level of the droplets did not exceed 100 μL. Then 100 μL of mineral oil (Sigma, M5904) was added to cover the surface of the droplets, and the 8-tube strip cover was closed to perform PCR according to the following procedure:
heating at 95° C. for 3 min;
heating at 98° C. for 30 s;
performing the following steps for 15 cycles
{heating at 98° C. for 10 s;
annealing at 58° C. for 30 s; and
extending at 72° C. for 1.5 min;}
heating at 72° C. for 5 min; and
maintaining at 4° C.

### Demulsification

After PCR, the droplets were transferred to a new low-adsorption 1.5 mL centrifuge tube (Invitrogen, AM12400), 50 μL of demulsifier perfluorooctanol (Sigma #370533) was added to dissolve the droplets, and the magnetic beads in the droplets were recovered. It was inverted and mixed evenly, and after being centrifuged at 1000 g for 2 min, it was placed on a magnetic rack, and stilly placed for 5 min. The magnetic bead adsorption state was maintained, the supernatant was discarded, and 1 mL of TE-TW was added, which was prepared by mixing TE buffer (Invitrogen, AM9849) with Tween-20 (Sigma-Aldrich, 9005-64-5) to a final concentration of 0.05%, and it was inverted and mixed evenly. The resulting solution was centrifuged under the condition of 1000 g for 2 min, then placed on a magnetic rack, and stilly placed for 2 min. The supernatant was discarded. The above steps were repeated twice.

A 200 μL enzyme digestion reaction system: 20 μL of 10×EXO I buffer, 10 μL of EXO I exonuclease (Thermo Scientific, EN0581), and 170 μL of water, was added; the system was incubated on a metal bath thermostat (Eppendorf, 5382000074) under shaking conditions of 37° C. and 1000 rpm for 45 min. After the reaction, it was centrifuged briefly, and 1 mL of TE-SDS was added, which was prepared by mixing the TE buffer (Invitrogen, AM9849) with SDS (Invitrogen, AM9820) to a final concentration of 0.5%, it was inverted and mixed evenly, and the reaction was terminated.

It was centrifuged under the condition of 1000 g for 2 min, then placed on the magnetic rack, and stilly placed for 2 min. The supernatant was discarded. 1 mL of TE-TW was added again, it was inverted and mixed evenly, centrifuged under the condition of 1000 g for 2 min, then placed on the magnetic rack and stilly placed for 1 min, and the supernatant was discarded; and the above steps were repeated once.

cDNA Amplification

The magnetic bead adsorption state was maintained, and 8 parts of the following prepared PCR system (800 μL in total): 50 μL of 2×KAPA mixture (Roche, 07958935001), 4 μL of 0.4 uM Tn primer (Sequence: 5′-CGTAGCCATGTCGTTCTG-3′), 18 μL of 10% Opti-prep (Sigma, D1556-250ML), and 28 μL of H₂O, were added. The magnetic beads were evenly mixed by blowing and sucking, then sub-packaged in the 8-tube strip at 100 μL/tube.

PCR was performed according to the following procedure:
heating at 98° C. for 30 s;
performing the following steps for 20 cycles:
{heating at 98° C. for 10 s,
annealing at 58° C. for 30 s, and
extending at 72° C. for 1.5 min;}
heating at 72° C. for 5 min; and
maintaining at 4° C.

After the reaction was terminated, 0.6X Vazyme DNA purification magnetic beads (Vazyme, #N411) were added to purify a PCR product, and finally 50 μL of NF-H₂O was used to elute cDNA.

Construction of cDNA Library

100-200 ng (approximately 0.1-0.2 pmol) of cDNA to be fragmented (broken) was taken, and placed in a new 0.2 mL PCR tube. The volume should be ≤16 μL, and the portion less than 16 μL was supplemented with H₂O. Fragmentation reaction solution was prepared on ice according to Table 1, a NEBNext® dsDNA Fragmentase and its buffer (NEB, M0348S) were used:

TABLE 1
Components of fragmentation reaction solution:

| Component | Volume | |
|---|---|---|
| Fragmentase buffer | 2.0 μL | 1X |
| Fragmentase | 2.0 μL | / |
| Total | 4.0 μL | / |

The PCR tube was placed on a PCR instrument, the heat cover was set to 75° C., and it was incubated at 37° C. for 10 min to obtain a fragmented product. After the reaction was completed, 30 μL of 0.1 M EDTA was added to the PCR tube, it was mixed evenly by vortex oscillation, and the reaction was terminated. 0.6×+0.2×Vazyme DNA purification magnetic beads (Vazyme, #N411) were used to purify and screen the fragmented product, and a qubit dsDNA high-sensitivity detection kit (Thermo, Q33230) was used for concentration quantification.

End repair reaction solution was prepared on ice according to Table 2, Roche's ER&A tailing enzyme and its buffer (Roche, KK8500) were used:

TABLE 2
Components of end repair reaction solution:

| Component | Volume |
|---|---|
| ER&A tailing buffer | 8.5 μL |
| ER&A tailing enzyme | 1.5 μL |
| Total | 10 μL |

10 μL of the prepared end repair reaction solution was sucked with a pipette and added to the fragmented product. After being evenly mixed by brief vortex, it was centrifuged instantaneously. Then, the PCR tube was placed on a PCR instrument, the heat cover was set to 75° C., it was incubated at 37° C. for 30 min, heated at 65° C. for 15 min, and maintained at 4° C. to obtain an end repair product.

Linker linkage reaction solution was prepared on ice according to Table 3, NEB's ligase buffer A (NEB, M0202S) and DNA ligase (NEB, M0202S) were used:

TABLE 3
Components of linker linkage reaction solution:

| Component | Volume | |
|---|---|---|
| Ligase buffer A | 23.4 | μL |
| 10 μM linker (iDrop) | 5.0 | μL |
| DNA ligase | 1.6 | μL |
| Total | 30 | μL |

30 μL of the prepared linker linkage reaction solution was slowly sucked with the pipette, and added to the end-repair product. It was evenly mixed by vortex oscillation, and the reaction solution was collected at the bottom of the tube by instantaneous centrifugation. The PCR tube was placed on a PCR instrument, the heat cover was set to 75° C., and it was incubated at 23° C. for 30 min, and then maintained at 4° C. to obtain a linker linkage product.

1.0×Vazyme DNA purification magnetic beads (Vazyme, #N411) were used to purify the linker linkage product, and the qubit dsDNA high-sensitivity detection kit (Thermo, Q33230) was used to determine the concentration of the linkage product.

A linker primer PCR amplification reaction mixture was prepared in a centrifuge tube according to Table 4, and linker primer PCR amplification was performed:

TABLE 4
Components of linker primer PCR amplification reaction mixture:

| Component | Volume | |
|---|---|---|
| 2X KAPA HiFi Hotstart Ready mixture | 50 | μL |
| F primer (10 μM) | 2 | μL |
| R primer (10 μM) | 2 | μL |
| Linkage purified product | 46 | μL |
| Total | 100 | μL |

F primer: 5′-PhoCGTAGCCATGTCGTTCTG*C *-3′, wherein PhoC at the 5′ end was C with phosphorylation modification, and the two bases G*C* at the 3′ end were with thio-modification.

R primer:
5′-TGTGAGGCCAAGGTGTTGTCTTCCTAAGACCGCT-3′.

54 μL of the prepared linker primer PCR amplification reaction mixture was sucked with a pipette and added to the purified linker linkage product, it was evenly mixed by vortex oscillation, and the reaction solution was collected at the bottom of the tube by instantaneous centrifugation. Linker primer PCR amplification was performed according to the following scheme:
95° C. for 3 min;
performing the following steps for 10-15 cycles:
{heating at 98° C. for 20 s,
annealing at 60° C. for 20 s, and
extending at 72° C. for 30 s;}
heating at 72° C. for 5 min; and
maintaining at 4° C.

After the linker primer PCR reaction was completed, 0.6×+0.6×Vazyme DNA purification magnetic beads (Vazyme, #N411) were used to purify a product, and a qubit dsDNA high-sensitivity detection kit (Thermo, Q33230) was used for product quantification.

### High-Throughput Sequencing

Before sequencing, the obtained cDNA product needed to be denatured. 200-400 ng of the cDNA product obtained above was taken, the volume was supplemented to 47 μL with NF-H₂O, 3 μL of 20 μM splint oligo (5′-GACATGGCTACGTGTGAGCCAAGG-3′) was added, it was evenly mixed by brief vortex and instantaneously centrifuged for 5 s. Then, the PCR tube was placed on a PCR instrument, the heat cover was set to 105° C., it was heated at 95° C. for 3 min, and then quickly placed on ice for 5-10 min.

A single-stranded cyclization reaction system was prepared according to Table 5, and NEB's DNA ligase and its ligase buffer B (NEB, M0202L) were used:

TABLE 5
Components of single-stranded cyclization reaction solution:

| Component | Volume |
|---|---|
| Ligase buffer B (NEB, M0202L) | 9.8 μL |
| DNA ligase (NEB, M0202L) | 0.2 μL |
| Total | 10 μL |

The prepared single-stranded cyclization reaction solution was added to the denatured product (namely a melt product), it was evenly mixed by brief vortex, and centrifuged instantaneously for 5 s, then the PCR tube was placed on a PCR instrument, the heat cover was set to 75° C., and it was incubated at 37° C. for 30 min.

At the end of the single-stranded cyclization reaction, enzymatic digestion reaction solution was prepared on ice in advance according to Table 6, and NEB's exonuclease B and its buffer (NEB, M0293S) were used:

TABLE 6
Components of enzymatic digestion reaction solution:

| Component | Volume |
|---|---|
| Exonuclease B | 2.6 μL |
| Exonuclease buffer B | 1.4 μL |
| Total | 4.0 μL |

4 μL of the prepared enzymatic digestion reaction solution was sucked with a pipette and added to the single-stranded cyclization product, it was evenly mixed by brief vortex, and centrifuged instantaneously for 5 s. Then, the PCR tube was placed on a PCR instrument, the heat cover was set to 75° C., and it was incubated at 37° C. for 30 min.

After the enzymatic digestion reaction was completed, 3 μL of termination solution (0.1 M EDTA) was added to the PCR tube, it was evenly mixed by vortex, and centrifuged instantaneously for 5 s, and the liquid was collected at the bottom of the tube.

2.5×Vazyme DNA purification magnetic beads (Vazyme, #N411) were used to purify the cyclization product, and a qubit dsDNA high-sensitivity detection kit (Thermo, Q33230) was used to quantify the purified product. When the library mass was >0.5 ng/μL, it was considered that the library detection was qualified and might be used for sequencing by a sequencer.

### Example 2: Ultra-High-Throughput Single-Cell Transcriptome Sequencing With Human-Derived 293T Cell Line

In this embodiment, ultra-high-throughput single-cell transcriptome sequencing was performed with a human-derived 293T cell line. The human-derived 293T cell line was fixed with methanol, and 24 types of barcodes were used in the first-round indexing. The value of reads in cell was: 0.769, the average number of UMI of each cell was: 1008, and the mean gene number of each cell was: 800. Finally, nearly 20000 usable cells were obtained with a single chip.

### Example 3: Ultra-High-Throughput Single-Cell Transcriptome Sequencing With Mixed Cell Lines of Human-Derived 293T and Mouse-Derived 3T3

In this embodiment, ultra-high-throughput single-cell transcriptome sequencing was performed with mixed cell lines of human-derived 293T and mouse-derived 3T3. The mixed cell lines of human-derived 293T and mouse-derived 3T3 was fixed with methanol, and 48 types of barcodes were used in the first-round indexing. The value of reads in cell was: 0.626, the average number of UMI of each cell was: 1127, and the mean gene number of each cell was: 469. Finally, approximately 30000 usable cells were obtained with a single chip. The doublet rate of human and mouse cells was 1.59%.

### Example 4: Ultra-High-Throughput Single-Cell Transcriptome Sequencing Using Mixed Cell Lines of Human-Derived 293T and Mouse-Derived 3T3

In this embodiment, ultra-high-throughput single-cell transcriptome sequencing was performed with mixed cell lines of human-derived 293T and mouse-derived 3T3. The mixed cell lines of human-derived 293T and mouse-derived 3T3 was fixed with methanol, and 96 types of barcodes were used in the first-round indexing. The value of reads in cell was: 0.766, the average number of UMI of each cell was: 661, and the mean gene number of each cell was: 104. Finally, approximately 80000 usable cells were obtained with a single chip. The doublet rate of human and mouse cells was 10.65%.

From the above description, it could be seen that the method of the present invention achieves the ultra-high-throughput single-cell transcriptome sequencing of the cells from different cell lines and tissue sources, specifically as follows:
1) The method of the present invention achieves the overloading of the cells or cell nuclei after the in-situ reverse transcription together with the pretreated magnetic beads, and an efficient, flexible, easy-to-use and large-scale scRNA sequencing method is provided. The first-round indexing could further expand the throughput by increasing the types of the first-round barcodes. Since each well in the first-round indexing receives a unique barcode, hundreds or even thousands of individual samples could be directly analyzed in parallel in a single experiment-such as measuring transcriptome responses to a large amount of genetic or pharmacological perturbation in the cell lines, or treating tissue samples from large epidemiological cohorts.
2) The method of the present invention is simple in operation, high in efficiency, and achieves the ultra-high throughput of 100000-cell level in a single experiment. This method combines the scalability of the combinatorial indexing with the efficiency and usability of a droplet generator in a simple two-stage workflow. The present invention shows a 10-fold increase in throughput compared to a regular droplet-seq experiment in a single experiment. Experimental devices required by the present invention are simple, and only the standard laboratory device and the microfluidic droplet generator are required. Only two rounds of indexing are required, the reverse transcription could be completed in the intact cells or cell nuclei.

The above are only preferred embodiments of the present invention and are not intended to limit the present invention. For those skilled in the art, the present invention may have various modifications and variations. Any modifications, equivalent replacements, improvements and the like made within the spirit and principles of the present invention shall be contained within the scope of protection of the present invention.

What is claimed is:

1. A construction method for a single-cell transcriptome sequencing library, wherein the construction method comprises the following steps:
1. performing in-situ reverse transcription in a cell or a cell nucleus on single-cell suspension to generate a full-length first chain cDNA with a first barcode;
2. overloading the cell or the cell nucleus obtained in Step 1) after the in-situ reverse transcription and a polymerase chain reaction (PCR) system used for second chain cDNA synthesis together with a magnetic bead capable of recognizing the first barcode into a droplet, and performing a droplet PCR;
3. recovering the magnetic bead after the droplet PCR;
4. performing secondary amplification on cDNA on the magnetic bead obtained in Step 3); and
5. constructing a fragmentation library of cDNA obtained in Step 4) after the secondary amplification to obtain the single-cell transcriptome sequencing library.

2. The construction method according to claim 1, wherein in Step 1), an indexed oligo-dT primer is used for the in-situ reverse transcription to generate the full-length first chain cDNA with the first barcode;
wherein, the oligo-dT primer sequentially comprises from 5′ to 3′:
a. a magnetic bead capture region, which is a complementary region complementary to an oligonucleotide in a second barcode on the magnetic bead;
b. a unique molecular identifier used to recognize a single cDNA transcript;
c. a first-round barcode used to distinguish different cells or cell nuclei; and
d. a poly T sequence used to capture an mRNA;
thus, the 5′ end of the obtained full-length first chain cDNA with the first barcode sequentially comprises from 5′ to 3′: the magnetic bead capture region, which is the complementary region complementary to the oligonucleotide in the second barcode on the magnetic bead; the unique molecular identifier; and the first-round barcode and the poly T sequence, wherein the magnetic bead is capable of recognizing the magnetic bead capture region in the first barcode.

3. The construction method according to claim 1, wherein in Step 1), the in-situ reverse transcription is performed with a reverse transcriptase having terminal transferase activity, and the in-situ reverse transcription system further comprises a template switch oligo primer (Tn-TSO).

4. The construction method according to claim 1, wherein the in-situ reverse transcription system further comprises glycerol.

5. The construction method according to claim 1, wherein in Step 2), the density of the cells or cell nuclei in the single-cell suspension is controlled to be 100˜1000 per μL, and the density of the magnetic beads is controlled to be 2000˜5000 per μL.

6. The construction method according to claim 1, wherein in Step 2), the PCR system for second chain cDNA synthesis comprises: a primer simultaneously complementary to the 3′ end of the full-length first chain cDNA and a magnetic bead oligo primer, as well as a PCR reagent;
wherein the primer simultaneously complementary to the 3′ end of the full-length first chain cDNA and the magnetic bead oligo primer is a Tn primer.

7. The construction method according to claim 1, wherein in Step 2), the magnetic bead is a magnetic bead with a second barcode.

8. The construction method according to claim 1, wherein Step 5) comprises:
constructing a fragmentation library of cDNA obtained in Step 4) after secondary amplification to obtain a linear cDNA library, wherein the linear cDNA library is the single-cell transcriptome sequencing library.

9. A sequencing method for a single-cell transcriptome library, wherein the sequencing method comprises:
constructing a single-cell transcriptome sequencing library by using the construction method according to claim 1; and
performing sequencing by a sequencer on the single-cell transcriptome sequencing library.

10. A kit for preparing a single-cell transcriptome library, wherein the kit comprises at least one of the following: an indexed oligo-dT primer with a first barcode, a template switch oligo primer, a reverse transcriptase, glycerol, an immobilization and/or permeabilization reagent, a primer simultaneously complementary to a 3′ end of a full-length first chain cDNA and a magnetic bead oligo primer, a magnetic bead with a second barcode, reagents for a linear cDNA library construction, and reagents for a circular cDNA library construction;
wherein, the indexed oligo-dT primer sequentially comprises from 5′ to 3′:
a. a magnetic bead capture region, which is a complementary region complementary to an oligonucleotide in the second barcode on the magnetic bead;
b. a unique molecular identifier used to recognize a single cDNA transcript;
c. a first-round barcode used to distinguish different cells or cell nuclei; and
d. a poly T sequence used to capture an mRNA.

11. The construction method according to claim 3, wherein in Step 1), the reverse transcriptase having terminal transferase activity is selected from Maxima H enzyme, SSII enzyme, or SSIV enzyme.

12. The construction method according to claim 3, wherein in Step 1), the template switch oligo primer is designed to comprise guanine located at a 3′-end.

13. The construction method according to claim 4, wherein the final concentration of the glycerol in the in-situ reverse transcription system is 5%˜10%.

14. The construction method according to claim 4, wherein the construction method further comprises performing immobilization and/or permeabilization pretreatment on the cell or the cell nucleus in the single-cell suspension before the in-situ reverse transcription.

15. The construction method according to claim 5, wherein in Step 2), the density of the cells or cell nuclei is 500 per μL, and the density of the magnetic beads is 3000 per μL.

16. The construction method according to claim 7, wherein in Step 2), the second barcode, according to a distance from the magnetic bead, sequentially comprises from near to far: the Tn primer, a second-round barcode, and a cDNA capture sequence for recognizing and capturing the full-length first chain cDNA.

17. The construction method according to claim 16, wherein the second-round barcode comprises a second-round first barcode and a second-round second barcode.

18. The construction method according to claim 17, wherein a first linker is further comprised between the second-round first barcode and the second-round second barcode, and a second linker is further comprised between the Tn primer and the magnetic bead.

19. The construction method according to claim 8, wherein in Step 5), further preparing the non-circular cDNA library into a circular cDNA library, wherein the circular cDNA library is the single-cell transcriptome sequencing library; wherein the step of preparing the non-circular cDNA library into the circular cDNA library comprises:
melting a double-chain cDNA in the non-circular cDNA library under the action of a cyclization auxiliary sequence to obtain a melt product;
ligating the melt product with a DNA ligase to obtain a single-chain cyclization product;
performing enzymatic digestion on the single-chain cyclization product to degrade the remaining non-cyclized single-chain cDNA and double-chain cDNA to obtain the circular cDNA library.

20. The kit according to claim 10, which is characterized by one or more of the following:
(i) the reverse transcriptase is a reverse transcriptase having terminal transferase activity;
(ii) the reverse transcriptase having terminal transferase activity is selected from Maxima H enzyme, SSII enzyme, or SSIV enzyme;
(iii) the template switch oligo primer is designed to comprise guanine;
(iv) the final concentration of the glycerol in the in-situ reverse transcription system is 5%˜10%;
(v) the immobilization and/or permeabilization reagent is methanol or paraformaldehyde, the working concentration of the methanol is 50%˜100%, and the working concentration of the polyformaldehyde is 1%˜4%;
(vi) the primer simultaneously complementary to the 3′ end of the full-length first chain cDNA and the magnetic bead oligo primer is a Tn primer;
(vii) in the magnetic bead with the second barcode, the second barcode, according to a distance from the magnetic bead, sequentially comprises from near to far: a Tn primer, a second-round barcode, and a cDNA capture sequence for capturing the first chain cDNA;
(viii) the second-round barcode comprises a second-round first barcode and a second-round second barcode; a first linker is further comprised between the second-round first barcode and the second-round second barcode; and a second linker is further comprised between the Tn primer and the magnetic bead;
(ix) the reagents for a linear cDNA library construction are used for reverse transcription and fragmentation library construction of amplified cDNA to obtain a linear cDNA library;
(x) the reagents for a circular cDNA library construction are used to prepare the linear cDNA library into a circular cDNA library.
